(19) United States
(12) Patent Application Publication
(10) Pub. No.: US 2026/0224821 A1
(43) Pub. Date: Aug. 6, 2026

(54) PARTICLE PREVENTION ADAPTER SYSTEM FOR INJECTION PEN

(71) Applicant: Takeda Pharmaceutical Company Limited, Osaka (JP)

(72) Inventor: Joyce Zhao, Acton, MA (US)

(21) Appl. No.: 18/723,698

(22) PCT Filed: Jan. 5, 2023
(86) PCT No.: PCT/IB2023/050080

§ 371 Date: Jun. 24, 2024

Related U.S. Application Data
(60) Provisional application No. 63/297,063, filed on Jan. 6, 2022.

(51) Int. Cl. A61M 5/34; A61M 5/24; A61M 5/31

(52) U.S. Cl. CPC A61M 5/345; A61M 2005/2407; A61M 2005/3104

(57) ABSTRACT

An adapter system for a pharmaceutical dispensing device having a connector that receives a septum of a pharmaceutical cartridge includes an adapter body and an adapter needle. The adapter body is configured to be slideably received within the connector of the pharmaceutical dispensing device. The adapter body includes a neck portion and a head portion extending from the neck portion, the neck portion has a first diameter and an end surface configured to face the septum of the pharmaceutical cartridge when the adapter body is received within the connector. The head portion has a second diameter different than the first diameter. The adapter needle extends from the end surface of the neck portion, away from the adapter body, and is configured to pierce through the septum of the pharmaceutical cartridge when the adapter body is received within the connector.

### CROSS REFERENCE TO RELATED APPLICATIONS

This application claims priority under 35 U.S.C. § 119(e) to U.S. Provisional Application 63/297,063, filed on Jan. 6, 2022. The disclosure of this prior application is considered part of the disclosure of this application and are hereby incorporated by reference in its entirety.

### TECHNICAL FIELD

This disclosure relates to an adapter system for an injection pen.

### BACKGROUND

Multi-use injection pens are often used as medication delivery systems. These injection pens typically include a housing configured to receive a pharmaceutical cartridge, which may be provided with a resilient septum for sealing an open end of the pharmaceutical cartridge. In use, a double-ended pen needle is attached to an end of the injection pen for administrating a dose of the pharmaceutical contained within the cartridge. Common pen needles often include a single cannula extending through an attachment hub such that a non-patient (non-injection) end of the cannula pierces the septum each time the attachment hub is installed on the injection pen. Thus, each time the pen needle is installed and uninstalled, the cannula is inserted and removed from the septum. Over the course of multiple uses, the integrity of the septum may be degraded due to the repeated cycles of installation and removal of the needle.

### SUMMARY

The present disclosure provides an adapter system configured for use with a pen needle and an injection pen. Generally the adapter system includes an adapter that is configured to be attached to an end connector of an injection pen and provides a conduit for inserting a cannula of the pen needle through a septum of a pharmaceutical cartridge disposed within the injection pen. Particularly, the adapter has an adapter body configured to slidingly fit within the connector of the injection pen and an adapter needle that extends from the adapter body and is configured to pierce the septum. In use, the adapter remains installed within the connector over the multiple doses provided by the pharmaceutical cartridge and provides a conduit through which the cannula of the pen needle can pass through the septum into the pharmaceutical cartridge. By providing this conduit through the septum, the number of installation cycles (i.e., number of times a needle is inserted and removed from the septum) can be reduced to a single installation, thereby preventing material degradation of the septum. The adapter system further includes a removable cap configured to fit on or over the end connector of the injection pen when the pen needle is uninstalled from the injection pen. Here, the cap seals the conduit through the septum between uses to preserve the pharmaceutical within the pharmaceutical cartridge.

An aspect of the disclosure relates to an adapter system for a pharmaceutical dispensing device having a connector that receives a septum of a pharmaceutical cartridge. The adapter system includes an adapter body configured to be slideably received within the connector of the pharmaceutical dispensing device, the adapter body including a neck portion and a head portion extending from the neck portion, the neck portion having a first diameter and an end surface configured to face the septum of the pharmaceutical cartridge when the adapter body is received within the connector, the head portion having a second diameter different than the first diameter. The adapter system also includes an adapter needle extending from the end surface of the neck portion away from the adapter body and configured to pierce through the septum of the pharmaceutical cartridge when the adapter body is received within the connector.

This aspect of the disclosure may include one or more of the following optional features. In some implementations, the adapter needle has a constant diameter. In some examples, the head portion is cylindrical. In some configurations, the neck portion is cylindrical. In some implementations, the neck portion is conical. In some examples, the adapter body includes a shoulder portion disposed between the head portion and the neck portion, the shoulder portion tapering from the head portion to the neck portion.

In some examples, the adapter system further includes an adapter conduit extending through the adapter body and the adapter needle. In some configurations, the adapter conduit terminates at a tapered opening formed in the adapter body. In some implementations the adapter conduit is sized to provide a clearance fit for a needle size associated with the adapter system. In some examples, the adapter body is made from a first material and the adapter needle is made from a second material different than the first material.

In some implementations, the adapter system includes a cap configured to attach to the connector of the pharmaceutical dispensing device when the adapter body is received within the connector. In some examples, the cap is configured to seal the connector. In some implementations, the cap includes an end wall and a peripheral sidewall extending from the end wall to define a cavity configured to frictionally receive the connector. In some examples, the peripheral sidewall of the cap includes a retention feature configured to secure the cap to the connector.

Another aspect of the disclosure provides an adapter system for a pharmaceutical dispensing device. The adapter system includes an adapter body including a head portion and a neck portion, the head portion of the adapter body having a first diameter and the neck portion having a second diameter different than the first diameter. The adapter system further includes an adapter needle extending from the neck portion of the adapter body to a distal end and having a third diameter that is less than each of the first diameter and the second diameter.

Aspects of the disclosure may include one or more of the following optional features. In some examples the third diameter is constant. In some implementations, the first diameter of the head portion is greater than the second diameter of the neck portion. In some configurations, the first diameter is constant. In some implementations, the second diameter is constant. In some implementations, the neck portion tapers down from the second diameter to a third diameter. In some examples, the adapter body includes a shoulder portion disposed between the head portion and the neck portion, the shoulder portion having a cross section that tapers from the first diameter to the second diameter.

In some examples, the adapter system includes an adapter conduit extending through the adapter body to the distal end of the adapter needle. Optionally, the adapter conduit includes a tapered opening defined at an end of the adapter body opposite the distal end of the adapter needle. In some examples, the adapter conduit is sized to provide a clearance fit for a needle size associated with the adapter system.

Another aspect of the disclosure provides a method of using a pharmaceutical dispensing device having a connector that receives a septum of a pharmaceutical cartridge. The method includes providing an adapter having (i) an adapter needle including a distal end and a proximal end; and (ii) an adapter body attached to the proximal end of the adapter needle and having a greater diameter than the adapter needle. The method also includes piercing the septum with the distal end of the adapter needle, wherein the distal end of the adapter needle is disposed within the pharmaceutical cartridge. The method also includes inserting the adapter body into the connector.

This aspect of the disclosure may include one or more of the following optional features. In some examples, inserting the adapter body into the connector includes inserting a neck portion of the adapter body into a throat of the connector and inserting a head portion of the adapter body into a mouth portion of the connector. In some configurations, the head portion of the adapter body has a greater diameter than the neck portion of the adapter body.

In some implementations, the method further includes attaching a cap to the connector to enclose the adapter within the connector. In some examples, the method further includes removing the cap from the connector. In some configurations, the method further includes installing a pen needle on the connector. In some implementations, installing the pen needle on the connector includes inserting a cannula of the pen needle through the adapter. In some examples, installing the pen needle on the connector includes inserting the cannula of the pen needle into the pharmaceutical cartridge through the adapter.

The details of one or more implementations of the disclosure are set forth in the accompanying drawings and the description below. These aspects and implementations, and other combinations from those described above, are indicative of various ways in which the subject matter may be practiced, all of which are intended to be within the scope of the claimed subject matter. Other aspects, features, and advantages will be apparent from the description and drawings, and from the claims.

### DESCRIPTION OF DRAWINGS

FIG. 1A is an exploded perspective view of an example adapter system configured for use with an injection device according to the principles of the present disclosure.

FIG. 1B is a perspective view showing the adapter system of FIG. 1A in a first configuration, wherein a connector cap of the adapter system is installed on the injection device.

FIG. 1C is a perspective view showing the adapter system of FIG. 1A in a second configuration, wherein a pen needle assembly is attached on the injection device in conjunction with an adapter of the adapter system.

FIG. 2 is an exploded cross-sectional view of the adapter system of FIG. 1A, taken relative to a longitudinal axis of the injection device.

FIG. 3A is an enlarged partial cross-sectional view of the adapter system of FIG. 2, taken at Area 3 in FIG. 2.

FIG. 3B is an enlarged partial cross-sectional view of the adapter system of FIG. 3A, showing the adapter system in a partially assembled configuration.

FIG. 4 is a cross-sectional perspective view of the adapter system of FIG. 1A, wherein a needle assembly is attached on the injection device in conjunction with an adapter of the adapter system.

FIG. 5 is a cross-sectional perspective view of the adapter system of FIG. 1A, wherein a connector cap of the adapter system is installed on the injection device.

FIG. 6 is a partial elevation view of another example of an adapter according to the present disclosure, where the adapter includes a cylindrical neck portion.

FIG. 7 is a partial elevation view of another example of an adapter according to the present disclosure, where the adapter includes a shoulder portion connecting a head portion and a neck portion.

FIG. 8 is a partial elevation view of another example of an adapter according to the present disclosure, where the adapter includes a radiused neck portion.

Like reference symbols in the various drawings indicate like elements.

### DETAILED DESCRIPTION

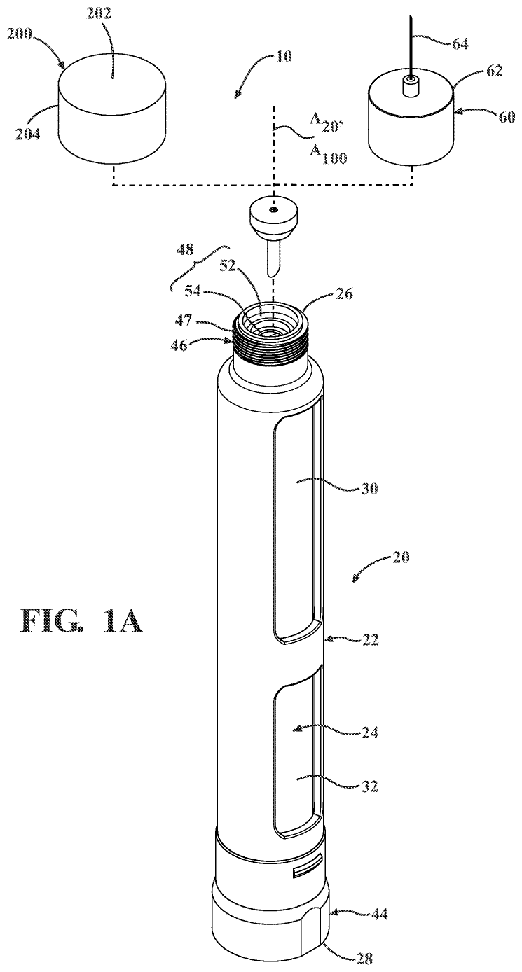

FIGS. 1A-5 illustrate an example of an adapter system 10 for use with a pharmaceutical injection device 20, also referred to as a “pen injector.” As described in greater detail below, the system 10 may be provided as a kit and includes an adapter 100 and a cap 200 that are each configured to be used in conjunction with the injection device 20 to allow the injection device 20 to be used over an extended period including several days or doses. The adapter system 10 may also be used with a pen needle 60 that can be interchangeably attached to the injection device 20. As described throughout, the pen needle 60 includes an attachment hub 62 and a cannula having a patient end 64 (i.e., the injection end) and a non-patient end 66 (i.e., the end extending away from the patient when in use).

FIGS. 1A-1C show the injection device 20 as an embodiment of a mechanical injection device in which the dispensing of a dosage of injection liquid is performed automatically. The injection device 20 includes a housing 22 and a cartridge 24 disposed within the housing 22. As shown in FIG. 1A, the injection device 20 extends along a longitudinal axis Azo from a proximal end 26 to a distal end 28. “Proximal” refers to that side of the injection device 20 that faces the pierced location of the patient during an injection, and “distal” refers to that side that faces away from the pierced location. The proximal direction describes the injection direction, or the direction in which the injection liquid is dispensed from a container. The distal direction describes the opposite direction.

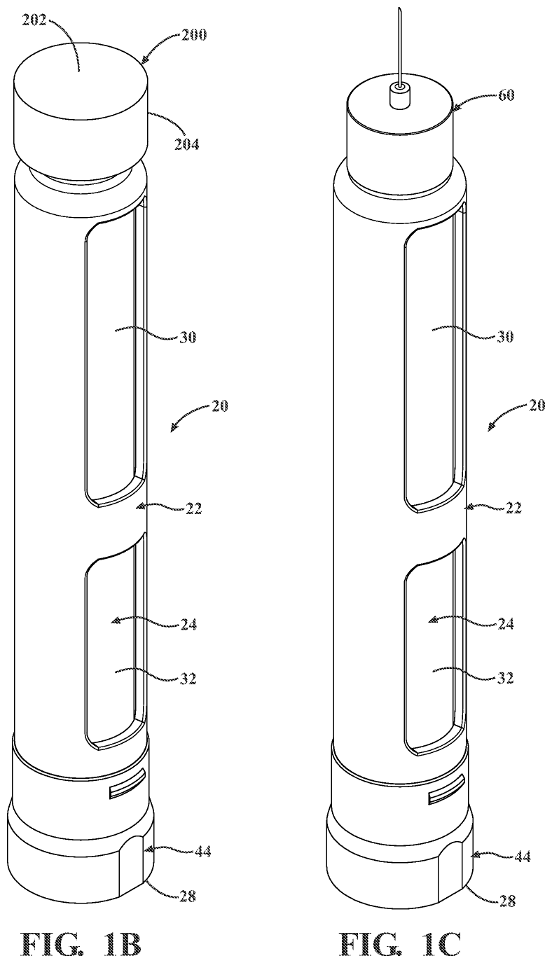

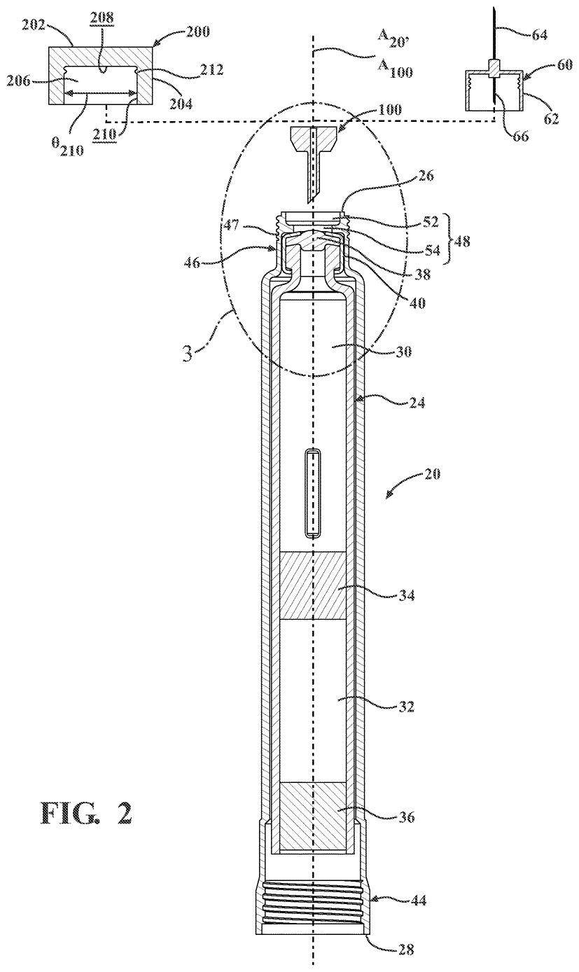

In the illustrated example, the cartridge 24 (see FIG. 2) is shown as a dual-chamber cartridge 24 including a first or proximal chamber 30 and a second or distal chamber 32. These chambers 30, 32 are separated by a sliding piston or stopper, namely a first stopper 34. The proximal end of cartridge 24 is sealed by a second sliding stopper 36. As shown in greater detail in FIG. 3B, the proximal end of proximal chamber 30 is sealed by pierceable membrane or septum 38 that is secured in place by a crimped metal cap 40. A solvent may be contained in the distal chamber 32 between the stoppers 34, 36 while a drug agent may contained in the proximal chamber 30. While a dual-chamber cartridge 24 is illustrated, the principles of the present disclosure apply to any cartridge 24 including at least one chamber and a resilient septum 38 sealing the chamber 24.

With reference to FIGS. 1A-3B, the housing 22 of the injection device 20 defines a cartridge holder 42 configured to accept and securely retain the cartridge 24 within the injection device 20. The housing 22 further includes an actuator 44 disposed on the distal end 28 of the housing 22. In the illustrated example, the actuator 44 is connectable in a rotationally fixed manner to the distal end 28 of the housing 22 and is configured to operate a dispensing mechanism (not shown) of the dispensing device 20 by rotating the actuator 44 about the longitudinal axis Ano relative to the housing 22. The actuator is operable to bias the stoppers 34, 36 of the cartridge 24 to reconstitute and dispense the drug agent and the solvent from the injection device 20.

The housing 22 further includes a connector 46 extending from the proximal end 26 of the housing 22. The connector 46 is generally configured to provide an attachment interface 47 between the housing 22 and the hub 62 of the injection needle 60. For example, a double-ended pen needle 60 is typically attached to the connector 46 through a snap fit, threading, Luer-Lok, or other secure attachment such that the patient end 64 of the double ended needle cannula can achieve fluid communication with a fluid contents contained within the cartridge 24 positioned within the cartridge holder 42.

With reference to FIG. 3A, the connector 46 includes a substantially cylindrical body disposed at the proximal end 26 of the housing 22 and defines an exterior connection interface 47 for attaching the needle 60. For example, the exterior connection interface 47 may include exterior helical threads or other means for attaching a pen needle 60. The connector 46 also defines an interior passageway extending from the proximal end 26 to the cartridge holder 42. Here, the interior passageway includes a socket 48 formed at the proximal end 26 and a cartridge receptacle 50 extending from the socket 48. The cartridge receptacle 50 is generally configured to receive a proximal end of the cartridge 24 including the septum 38 and the metal cap 40. More specifically, when the cartridge 24 is inserted within the cartridge holder 42, the septum 38 is exposed to the socket 48 of the connector 46 for piercing by a cannula, either of the needle 60 or the adapter 100.

Referring still to FIG. 3A, the socket 48 of the connector 46 may be described as including a socket mouth 52 and a socket throat 54. The mouth 52 of the socket 48 is formed immediately adjacent to the proximal end 26 of the connector 46 and provides an opening into the interior passageway of the connector 46. Here, a cross-section of the mouth 52 relative to the longitudinal axis A20 has a first size. In the illustrated example, the mouth 52 has a circular cross section such that the first size is provided as a first diameter 052. The throat 54 of the socket 48 extends between and connects the mouth 52 and the receptacle 50 of the connector 46. Optionally, a cross section of the throat 54 may be smaller than one or both of the receptacle 50 and the mouth 52. For example, where the throat 54 has a circular cross section, a second diameter 054 of the throat 54 is smaller than the first diameter 052 of the mouth 52. As shown in FIG. 3, when the cartridge 24 is inserted in the cartridge holder 42, the septum 38 is presented to the mouth 52 of the socket 48 through the throat 54 for piercing by a cannula or needle.

Turning now to the adapter system 10 or kit, an example of an adapter 100 configured for use with the injection device 20 is shown in various states of assembly in FIGS. 1A-5. The adapter 100 may be generally described as having a length extending along a longitudinal axis A100 (FIG. 3A) from a first, proximal end 102 to a second, distal end 104. In use, the adapter 100 is configured to be inserted into the socket 48 of the connector 46 such that the longitudinal axis A100 is substantially aligned with and parallel to the longitudinal axis A20 of the injection device 20.

The adapter 100 includes an adapter body 110, an adapter needle 112 extending from the adapter body 110, and an adapter conduit 114 extending continuously through the adapter 100 along the longitudinal axis A100. Generally, the adapter body 110 is configured to be slideably received within the socket 48 of the connector 46 when the adapter needle 112 is inserted into the cartridge 24 through the septum 38. As discussed below, the adapter conduit 114 of the adapter 100 provides an interface for the patient-end 66 of the cannula of the pen needle 60 to be repeatedly inserted through and removed from the septum 38 without degrading the material integrity of the septum 38. In other words, the adapter 100 eliminates direct contact between the needle 60 and the septum 38 during use.

The adapter body 110 is disposed at the proximal end 102 of the adapter 100 and includes a first, head portion 120 and a second, neck portion 122. The head portion 120 of the adapter body 110 is disposed at the proximal end 102 and is configured to be slideably received within the mouth 52 of the connector 46 when the adapter 100 is installed within the connector 46 (FIG. 3B). As shown, a cross section of the head portion 120 is defined by an outer surface 124 and has a size that is smaller than the size of the cross-section of the mouth 52 such that the head portion 120 provides a clearance fit with the connector 46. In the illustrated example, the head portion 120 of the adapter body 110 has a cylindrical shape defining a third diameter 0120. The third diameter 0120 is less than the mouth diameter 052, but greater than the throat diameter 054. Thus, when the adapter 100 is assembled within the connector 46, the head portion 120 is situated within the mouth 52 and provides radial clearance between the outer surface 124 and the connector 46 to accommodate misalignment of the adapter 100 within the connector 46 (e.g., radial or angular offset between the longitudinal axis A100 of the adapter and the longitudinal axis A20 of the injection device 20).

The neck portion 122 of the adapter body 110 include a second outer surface 126 that extends from the head portion 120 towards the distal end 104 of the adapter 100 and terminates at a base surface 128 facing the distal end 104, as best seen in FIG. 3A. As shown, a cross section of the neck portion 122 is defined by the outer surface 126 and has a size that is smaller than the size of head portion 120. In the illustrated example, the outer surface 126 of the neck portion 122 of the adapter body 110 defines a frustoconical shape that tapers from the third diameter 0120 of the head portion 120 to a fourth diameter 0122 that is less than the third diameter 0120. Here, the fourth diameter 0122 of the neck 122 is less than the throat diameter 054, which allows the neck portion 122 to be inserted through the throat 54 such that the base surface 128 is adjacent to or abutting the septum 38, as best seen in FIG. 3B.

With continued reference to FIG. 3A, the adapter body 110 has an overall length L110 corresponding to a height or depth of the socket 48. Thus, when the adapter 100 is properly installed within the dispensing device 20, the overall length L110 of the adapter body 110 is configured such that the proximal end 102 of the adapter is at or near the proximal end 26 of the dispensing device 20 defined by the connector 46. For example, the proximal end 102 of the adapter 100 is flush or slightly recessed from the proximal end 26 of the dispensing device 20. As discussed in greater detail below, this configuration allows the cap 200 to be attached to the connector 46 to seal the dispensing device 20 between uses (i.e., when the needle 60 is removed).

The overall length L110 of the adapter body 110 includes a length L120 of the head portion 120 extending from the proximal end 102 to the neck portion 122 and a length L122 of the neck portion 122 extending from the head portion 120 to the base surface 128. The length L120 of the head portion 120 may be less than or equal to a depth of the mouth 52 (measured from the proximal end 26 to the throat 54) such that the head portion 120 can be fully received within the mouth 52 and the proximal end 102 of the adapter 100 sits flush with or below the proximal end 26. The length L122 of the neck portion 122 may be configured such that the base surface 128 of the neck portion is positioned adjacent to or abuts a proximal surface (i.e., top) of the septum 38 when the adapter 100 is installed in the connector 46.

Referring still to FIGS. 1A-3B, a length L112 of adapter needle 112 extends along the longitudinal axis A100 of the adapter 100 from the base surface 128 of the neck portion 122 to a tip 130 formed at the distal end 104 of the adapter 100. As shown in FIG. 3A, the adapter needle 112 includes an outer surface 132 that extends from the base surface 128 to the tip 130 and defines an outer cross section of the adapter needle 112. In the illustrated example, the outer surface 132 of the adapter needle 112 is cylindrical in shape and defines a constant diameter 0112 from the base surface 128 to the tip 130. The tip 130 of the adapter needle 112 may be formed as a beveled surface defining a sharpened end of the adapter needle 112 for piercing the septum 38. In the illustrated example, the tip 130 includes a single beveled surface, but other examples of the tip 130 may include more than one beveled surface forming the piercing interface. The length L112 of the adapter needle 112 is configured to extend through the septum 38 and into the cartridge 24 when the adapter 100 is installed in the connector 46.

As previously discussed, the adapter 100 includes an adapter conduit 114 extending continuously through the adapter 100 from the proximal end 102 to the distal end 104. Generally, a cross section of the adapter conduit 114 is sized to provide a clearance fit with the patient end 64 of the cannula of the pen needle 60. For example, the adapter conduit 114 may have a cylindrical shape having a diameter 0114 that is larger than a diameter of the cannula of the pen needle 60. Optionally, the adapter conduit 114 may include an inlet 134 formed in the proximal end 102 of the head portion 120. The inlet 134 may include beveled (i.e., angled) or filleted (i.e., radiused) surface connecting the proximal end 102 and the adapter conduit 114, thereby providing a tapered opening for guiding a tip of the pen needle 60 into the adapter conduit 114.

As discussed above, the length L112 of the adapter needle 112 is configured to extend through the septum 38 and into the cartridge 24 when the adapter 100 is installed in the connector 46. Thus, the length L112 of the adapter needle 112 is at least the same as a thickness of the septum 38. However, the length L112 of the adapter needle 112 may be selected to be greater than the thickness of the septum 38 such that the adapter needle 112 extends substantially into a chamber of the cartridge 24 (i.e., beyond a neck of the cartridge). Here, the extended length of the adapter needle 112 provides a longer adapter conduit 114, which in turn, provides an increased alignment path for inserting the cannula of the pen needle 60. In other words, the needle 60 is prevented from being misaligned (e.g., off-center, skewed) with the longitudinal axis A20 of the injection device 20.

In the illustrated example, the adapter 100 is provided as a unitary component including the adapter body 110 and the adapter needle 112 formed as a single piece. Thus, the adapter body 110 and the adapter needle 112 may include the same material and be formed using a conventional molding process, such as injection molding. In other examples, the adapter 100 may be manufactured using additive manufacturing processes (i.e., three-dimensional printing), which allows the sizes, shapes, and materials of the adapter 100 to be customized to fit corresponding injection devices 20. In some examples, the adapter 100 may be constructed as a composite structure having an adapter body 110 including a first material and an adapter needle 112 including a second material that is different than the first material. For example, the adapter body 110 may include materials having a lower durometer than the materials of the adapter needle 112, thereby providing a relatively soft adapter body 110 configured to compress and conform to the shape of the receptacle 50 and a relatively rigid adapter needle 112 configured to pierce the septum 38.

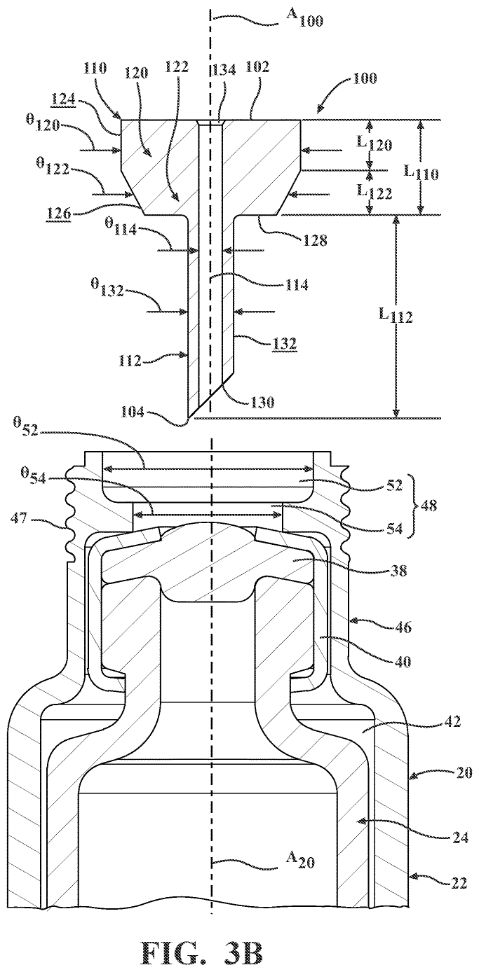

With continued reference to FIGS. 1A-1B and FIG. 5, the adapter system 10 further includes a connector cap 200 configured to be selectively attached to the connector 46 of the dispensing device 20 when the adapter 100 is installed, thereby sealing the connector 46 and the cartridge 24 to prevent inadvertent dispensing of the cartridge contents when the injection device is not in use. As best shown in FIGS. 2 and 5, the cap 200 includes an end wall 202 and a peripheral sidewall 204 extending from the end wall 202 to define a cavity 206 configured to receive the connector 46 of the injection device 20.

With continued reference to FIGS. 2 and 5, the interior cavity 206 of the connector cap 200 is defined by an end surface 208 of the end wall 202 and an inner surface 210 of the sidewall 204. In the illustrated example, the end surface 208 of the end wall 202 is a substantially flat surface and is configured to form a seal against the proximal end 26 of the connector 46 and/or the proximal end 102 of the adapter to prevent the contents of the cartridge 24 from escaping the injection device 20. While the illustrated example shows a flat end surface 208, the end surface 208 may also include a plug structure (not shown) configured to be inserted into the adapter conduit 114. For example, the plug structure may have a size and shape for frictionally inserting the plug into the inlet 134 of the adapter conduit 114 when the connector cap 200 is installed on the connector 46 (FIG. 5).

The inner surface 210 of the connector cap 200 is configured to provide a frictional fit with the exterior connection interface 47 of the connector 46. For example, where the connector 46 is formed as a cylindrical structure such as that shown in the figures, the inner surface 210 of the connector cap 200 may include an inner diameter 0210 that is less than an outer diameter of the connector 46 such that the frictional interface between the inner surface 210 of the connector cap 200 and the exterior connection interface 47 of the connector 46 provides sufficient friction to secure the connector cap 200 to the connector 46 while still allowing the connector cap 200 to be removed from the connector 46 by hand (i.e., without tools or damage to the connector 46 or cap 200).

In some configurations, the connector cap 200 may include a relatively rigid material, such as a metal or plastic material having an elastic modulus that is equal to or greater than an elastic modulus of a material of the connector 46. Thus, the connector cap 200 may be configured to form a slight interference fit with the connector 46 without deforming the connector cap 200. In some examples, the connector cap 200 may include a resilient material having an elastic modulus that is less than an elastic modulus of the material of the connector 46, such as a rubber material, which is configured to provide a sliding friction fit over the connector 46 when the connector cap 200 is pressed onto the connector 46. In other words, the connector cap 200 may be configured to stretch over the connector 46. Optionally, the inner surface 210 of the connector cap 200 may include one or more retention features 212 (FIGS. 2 and 5) configured to interface with the connector 46 to provide alignment and between the connector cap 200 and the connector 46 and to secure the connector cap 200 to the connector 46. For example, the connector cap 200 may include a retention rib 212 formed adjacent to the end surface 208 and circumscribing the cavity 206. In other examples, the inner surface 210 may include a plurality of ribs 212 or female helical threading configured to engage male helical threading formed on the exterior of the connector 46.

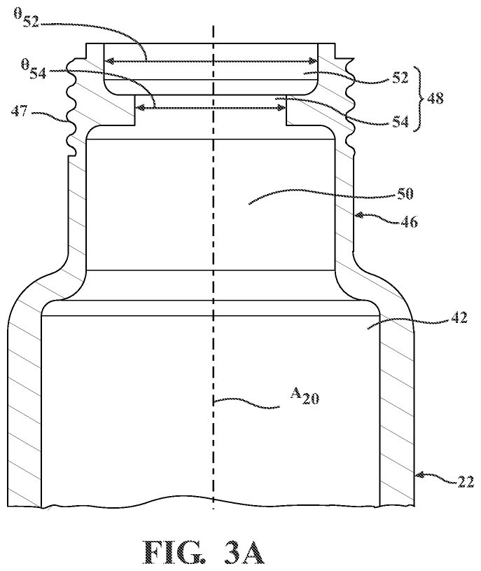

In use, the adapter system is operable to convert the injection device 20 between a dispensing state (FIGS. 1C and 4) and a storage state (FIGS. 1B and 5). With reference to FIGS. 1C and 4, the injection device 20 is configured in the dispensing state by installing the adapter 100 into the connector 46 of the injection device 20. Initially, the longitudinal axis A100 of the adapter 100 is aligned with the longitudinal axis A20 of the injection device 20 such that the tip 130 of the adapter needle 112 is aligned with the mouth 52 of the socket 48. The adapter needle 112 is then inserted through the septum 38 by piercing the septum 38 with the tip 130 of the adapter needle 112. Once the septum 38 is pierced, the adapter 100 is pressed in the distal direction such that the adapter body 110 seats within the socket 48 of the connector 46. More specifically, the neck portion 122 of the adapter body 110 is received within the throat 54 of the socket 48 such that the base surface 128 seats against or adjacent to the septum 38 and the outer surface 126 of the neck portion 122 is positioned within the throat 54. Likewise, the head portion 120 of the adapter body 110 is received within the mouth 52 such that the proximal end 102 of the adapter body 110 is flush with the proximal end 26 of the connector 46.

Once the adapter 100 is fitted within the connector 46, the injection device 20 may be converted between the dispensing state (FIGS. 1C and 4) and the storage state (FIGS. 1B and 5) by interchanging the pen needle 60 and the connector cap 200. For example, to install the pen needle 60, the non-patient end 66 of the cannula is aligned with and inserted through the adapter conduit 114. Once the non-patient end 66 is inserted within the adapter conduit 114, the hub 62 of the pen needle 60 can be attached to the connector by snapping or threading the hub 62 onto the exterior connection interface 47 of the connector 46.

To convert the dispensing device to the storage state (FIGS. 1B and 5), the process for installing the pen needle 60 is reversed and the pen needle 60 is detached from the connector 46 for storage or disposal. The connector cap 200 is then attached to the connector 46 while the adapter 100 remains installed within the connector 46. To attach the connector cap 200, the cavity 206 of the connector cap 200 is aligned over the connector 46 and the sidewall 204 of the connector cap is pressed or threaded over the connector 46 until the end surface 208 of the end wall 202 contacts the proximal end 26 of the connector 46 and/or the proximal end 102 of the adapter 100. Thus, the end surface 208 forms a sealing interface with the proximal end 26 of the connector and/or the proximal end 102 of the adapter 100 to prevent the contents of the cartridge 24 from escaping. The retention feature 212 of the connector cap 200 may also provide a sealing interface around an outer periphery of the connector 46.

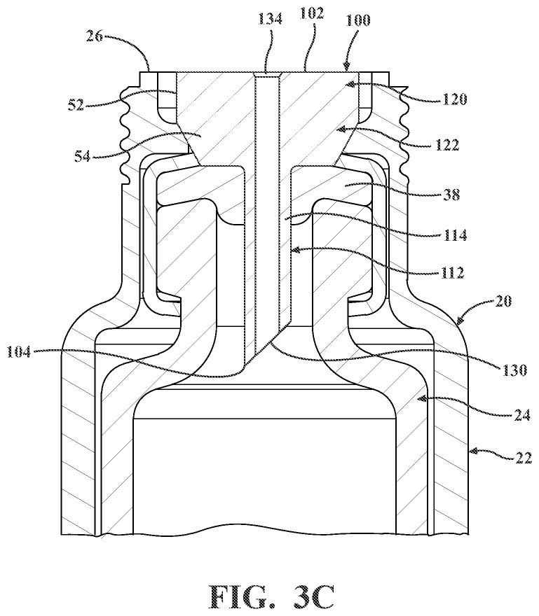

With reference to FIG. 6, another example of an adapter 100a is provided for use with the injection device 20 according to the present disclosure. In view of the substantial similarity in structure and function of the components associated with the adapter 100 with respect to the adapter 100a, like reference numerals are used hereinafter and in the drawings to identify like components while like reference numerals containing letter extensions are used to identify those components that have been modified.

The adapter 100a of FIG. 6 includes an adapter body 110a including the head portion 120 and a neck portion 122a. Unlike the neck portion 122 described previously, which included tapering cross section defining a frustoconical shape, the cross section of the neck portion 122a of the adapter 100a has a constant size extending from the head portion 120 to the base surface 128. For example, the neck portion 122a has a circular cross section having a constant diameter extending from the head portion 120 to the base surface 128 to provide the neck portion 122a with a cylindrical shape. A diameter of the neck portion 122a may be selected to provide a clearance fit, a slip fit, or a frictional fit with the throat 54 of the socket 48.

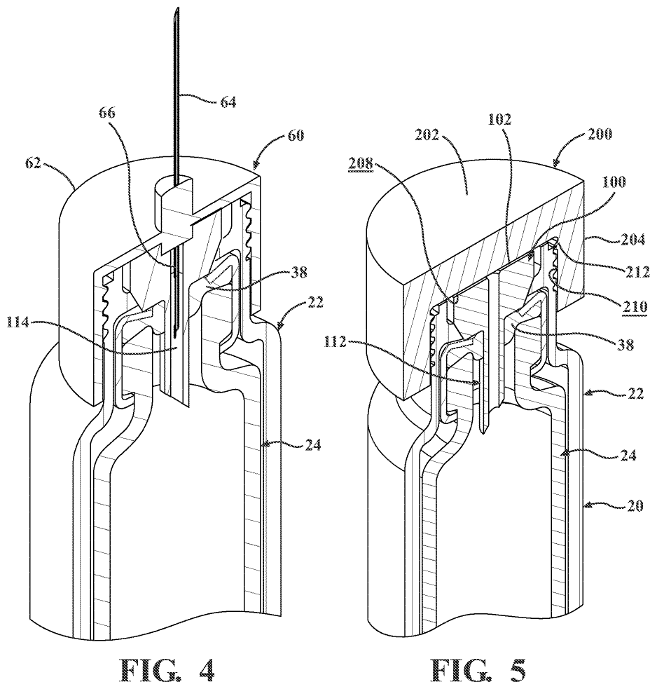

With reference to FIG. 7, another example of an adapter 100b is provided for use with an injection device 20 according to the present disclosure. In view of the substantial similarity in structure and function of the components associated with the adapter 100 with respect to the adapter 100b, like reference numerals are used hereinafter and in the drawings to identify like components while like reference numerals containing letter extensions are used to identify those components that have been modified.

The adapter 100b of FIG. 7 includes an adapter body 110b including the head portion 120, a neck portion 122b, and a shoulder portion 123 extending between and connecting the head portion 120 and the neck portion 122b. In this example, the head portion 120 is the same as the head portion 120 described previously. The neck portion 122b is disposed at the opposite end of the adapter body 110b from the head portion 120 and defines the base surface 128 of the adapter body 110b. Here, the neck portion 122b is cylindrical in shape and has a diameter that is less than the diameter of the head portion 120. The shoulder portion 123 of the adapter body 110b is disposed between and connects the cylindrical head portion 120 and the cylindrical neck portion 122b. As shown, the shoulder portion 123 has a conical shape that tapers from the diameter of the head portion 120 to the diameter of the neck portion 122b.

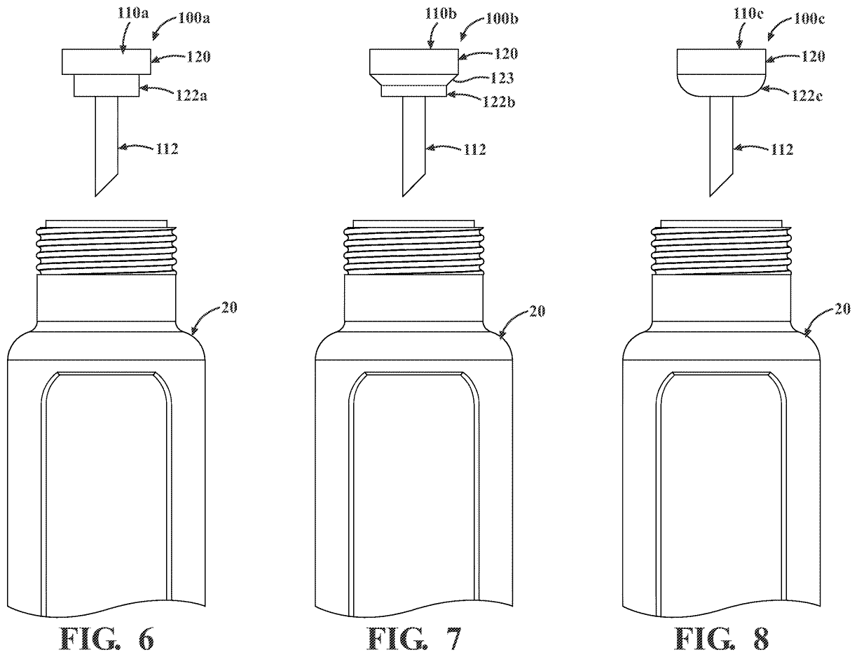

With reference to FIG. 8, another example of an adapter 100c is provided for use with an injection device 20 according to the present disclosure. In view of the substantial similarity in structure and function of the components associated with the adapter 100 with respect to the adapter 100c, like reference numerals are used hereinafter and in the drawings to identify like components while like reference numerals containing letter extensions are used to identify those components that have been modified.

The adapter 100c of FIG. 8 includes a body portion 110c including the head portion 120 and a neck portion 122c. Unlike the neck portion 122 described previously, which included a tapering cross section defining a conical shape, the cross section of the neck portion 122c of the adapter 100c has a tapering cross section defined by a radiused outer surface. Here, a radius of the outer surface of the neck portion 122b is selected to correspond to size of the throat 54 of the socket 48.

A number of implementations have been described. Nevertheless, it will be understood that various modifications may be made without departing from the spirit and scope of the disclosure. Accordingly, other implementations are within the scope of the following claims.

1. An adapter system for a pharmaceutical dispensing device having a connector that receives a septum of a pharmaceutical cartridge, the adapter system comprising:
an adapter body configured to be slideably received within the connector of the pharmaceutical dispensing device, the adapter body including a neck portion and a head portion extending from the neck portion, the neck portion having a first diameter and an end surface configured to face the septum of the pharmaceutical cartridge when the adapter body is received within the connector, the head portion having a second diameter different than the first diameter; and
an adapter needle extending from the end surface of the neck portion away from the adapter body and configured to pierce through the septum of the pharmaceutical cartridge when the adapter body is received within the connector.

2. The adapter system of claim 1, wherein the adapter needle has a constant diameter.

3. The adapter system of claim 1, wherein the head portion is cylindrical.

4. The adapter system of claim 1, wherein the neck portion is cylindrical.

5. The adapter system of claim 1, wherein the neck portion is conical.

6. The adapter system of claim 1, wherein the adapter body includes a shoulder portion disposed between the head portion and the neck portion, the shoulder portion tapering from the head portion to the neck portion.

7. The adapter system of claim 1, further comprising an adapter conduit extending through the adapter body and the adapter needle.

8. The adapter system of claim 7, wherein the adapter conduit terminates at a tapered opening formed in the adapter body.

9. The adapter system of claim 7, wherein the adapter conduit is sized to provide a clearance fit for a needle size associated with the adapter system.

10. The adapter system of claim 1, wherein the adapter body is made from a first material and the adapter needle is made from a second material different than the first material.

11. The adapter system of claim 1, further comprising a cap configured to attach to the connector of the pharmaceutical dispensing device when the adapter body is received within the connector.

12. The adapter system of claim 11, wherein the cap is configured to seal the connector.

13. The adapter system of claim 12, wherein the cap includes an end wall and a peripheral sidewall extending from the end wall to define a cavity configured to frictionally receive the connector.

14. The adapter system of claim 13, wherein the sidewall of the cap includes a retention feature configured to secure the cap to the connector.

15. An adapter system for a pharmaceutical dispensing device, the adapter system comprising:
an adapter body including a head portion and a neck portion, the head portion of the adapter body having a first diameter and the neck portion having a second diameter different than the first diameter; and
an adapter needle extending from the neck portion of the adapter body to a distal end and having a third diameter that is less than each of the first diameter and the second diameter.

16. The adapter system of claim 15, wherein the third diameter is constant.

17. The adapter system of claim 15, wherein the first diameter of the head portion is greater than the second diameter of the neck portion.

18. The adapter system of claim 15, wherein the first diameter is constant.

19. The adapter system of claim 15, wherein the second diameter is constant.

20. The adapter system of claim 15, wherein the neck portion tapers down from the second diameter to a third diameter.

21-32. (canceled)
